(19) United States
(12) Patent Application Publication
(10) Pub. No.: US 2004/0147463 A1
(43) Pub. Date: Jul. 29, 2004

(54) Anti-tumour anthracycline glycoside 14-sulfonic acid derivatives

(76) Inventors: Alessandro Arcari, Milan (IT); Mauro Gigli, Milan (IT)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/476,737

(22) PCT Filed: May 29, 2002
(86) PCT No.: PCT/EP02/05907

(30) Foreign Application Priority Data
Jun. 15, 2001 (GB) 0114654.7

(51) Int. Cl. A61K 31/704

(52) U.S. Cl. 514/34; 536/6.4

(57) ABSTRACT

Anthracycline glycosides of formula I, as reported in the description, wherein R is hydrogen, hydroxy or methoxy, R₁ and R₂, which are the same or different, are independently hydrogen or hydroxy, and the pharmaceutically acceptable salts thereof, are useful as anti-tumour agents. (I)

The invention relates to anthracycline glycosides, to a process for their preparation and to pharmaceutical compositions containing them.

The invention provides a compound which is an anthracycline glycoside of the formula I:

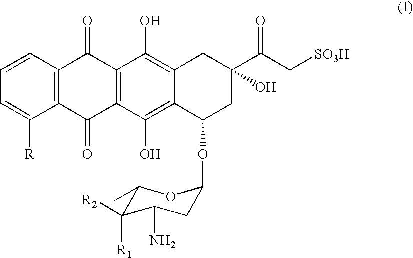

wherein R is hydrogen, hydroxy or methoxy, R₁ and R₂, which are the same or different, are independently hydrogen or hydroxy, or a pharmaceutically acceptable salt thereof.

In preferred compounds of formula I R is methoxy, R₁ is hydroxy and R₂ is hydrogen; or R is methoxy, R₁ is hydrogen and R₂ is hydroxy. These compounds are

daunorubicin 14-sulfonic acid (Ia) and

4′ epi daunorubicin 14-sulfonic acid (Ib).

The compounds of formula (I) may exist as zwitterions, i.e. as internal salts of a 14-SO₃-group with the amino group on the sugar residue; such salts are also part of the present invention.

The pharmaceutically acceptable salts are, for example, alkali metal and alkaline earth metal salts (e.g. sodium, potassium, lithium, calcium and magnesium salts), ammonium salts and salts with an appropriate organic amine or amino acid (e.g. arginine, procaine salts), and the addition salts formed with suitable organic or inorganic acids (e.g. hydrochlorides, hydrobromides, sulfates, phosphates) or carboxylic and sulfonic organic acids (e.g. acetates, citrates, succinates, malonates, lactates, tartrates, fumarates, maleates, methanesulphonates, p-toluenesulphonates).

The compounds of the formula I may be prepared by reacting the anthracycline of the formula II

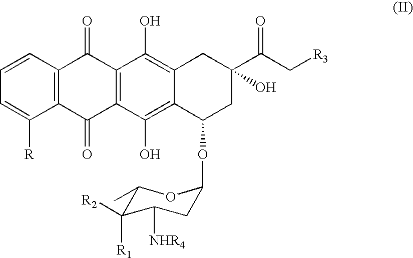

wherein R, R₁ and R₂ are as defined above, R₃ is hydroxy or a leaving group, and R₄ is hydrogen or a nitrogen protecting group, with a sulfiting agent in a polar solvent or a mixture of polar solvents, and, if desired, removing the protecting groups from the resulting compound. The resulting compound may be converted into a pharmaceutically acceptable salt thereof. Suitable leaving group which R₃ may represent include halogen atoms, such as bromine, iodine or chlorine, or mesyl, trifluoromesyl or p-toluensulfonyl groups. Suitable nitrogen protecting groups include, for instance, a trifluoroacetyl group. Examples of suitable solvents include acetone, water and mixtures of acetone and water. The reaction is preferably conducted at a temperature below 50° C., more preferably at room temperature. The desired resultant derivative may be isolated by precipitation from appropriate solvent mixtures. The starting material for the preparation of the new anthracycline glycosides is an optionally protected daunorubicin or 4-demethoxy daunorubicin, that may be easily converted into the compounds of the formula II as described in Antitumor Anthracyclines, Chim. Oggi, April 1990, p. 9-19.

The invention further provides a pharmaceutical composition comprising an anthracycline glycoside of formula I in admixture with a pharmaceutically acceptable diluent or carrier. Conventional carriers and diluents may be used. The composition may be formulated and administered in conventional manner.

The suspension or solutions for intramuscular injections may contain, together with the active compound, a pharmaceutically acceptable carrier, e.g. sterile water, olive oil ethyl oleate, glycols, e.g. propylene glycol and, if desired, a suitable amount of lidocaine hydrochloride. The solutions for intravenous injections or infusions may contain as carrier, for example, sterile water or preferably they may be in the form of sterile, aqueous, isotonic saline solutions or they may contain as a carrier propylene glycol.

The compounds according to the invention are useful in methods of treatment of the human or animal body by therapy. They are useful as anti-tumor agents. They are useful in the treatment of leukemia and solid tumors, such as colon, colon-rectal ovarian, mammary, prostate, lung, kidney and also melanoma tumors. A human can therefore be treated by a method comprising administering thereto a therapeutically effective amount of a compound of the invention. The condition of the human patient can thus be improved. The dosage to be given can be ascertained using known dosage ranges in the field of anthracyclines, modified by reference to the activity shown by the present compounds in in vitro and in vivo anti-tumor tests. Suitable dosages are generally in the range of 1 to 200 mg/m² body surface, preferably from 1 to 100 mg/m², depending on the nature and severity of the disease being treated and on the general condition of the patient.

The compounds of formula I were tested and found active in vivo against a tumor cell lines, and in vivo on murine leukemia.

On tumor cell lines, the compounds of the present invention were shown to present high cytotoxicity. The in vivo tests were carried on murine leukemia.

### EXAMPLE 1

Daunorubicin 14-sulfonic Acid (Ia)

14-bromo-daunorubicin hydrochoride (II, R=OCH₃, R₁=OH, R₂=H, R₃=Br, 2 mmol) were dissolved in acetone-water (30+30 ml). The solution was added with an aqueous solution of sodium sulfite (570 mg, 4.38 mmol), and the pH was adjusted to about 7.6 with aqueous ammonia 100%. After stirring 3 hours at room temperature, the reaction mixture was concentrated to dryness. The residue was suspended in methanol and the methanolic suspension was dropped in acetone at 4-5EC. The precipitate was filtered, washed on the filter and dried under vacuum. The product has been identified by MS and NMR analysis.

ESI (+) Mass spectrum

| m/z | Intensity | Attribution |
|---|---|---|
| 608 | 100 | [M + H]⁺ |
| 590 | 12 | [M − H₂O + H]⁺ |
| 461 | 10 | 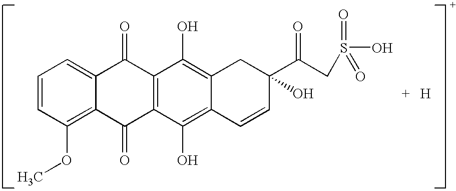 |
| 443 | 12 | 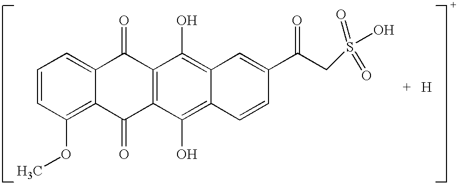 |
| 381 | 4 | 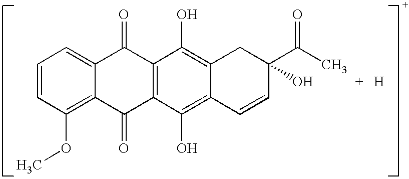 |
| 363 | 6 | 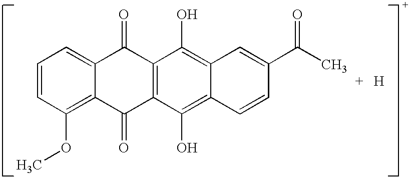 |
| 321 | 2 | 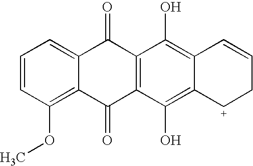 |

High resolution mass spectrometry.

An exact mass determination has been performed on the protonated molecular ion at m/z 608, with the following result

Calculated for C₂₇H₂₉NO₁₃S+H: 608.14379 Found: 608.14374 Δ=0.0 ppm

¹H NMR chemical shifts and coupling constants

| δ (ppm) | Multiplicity* | J (Hz) | Proton |
|---|---|---|---|
| 1.11 | d | 6.5 | CH₃-6′ |
| 1.62 | dd | 12.5, 4.2 | H-2′eq |
| 1.83 | ddd | 12.5, 12.5, 3.4 | H-2′ax |
| 2.03 | dd | 14.8, 5.3 | H-8β |
| 2.36 | d | 14.8 | H-8α |
| 2.73 | d | 18.0 | H-10β |
| 2.99 | d | 18.0 | H-10α |
| 3.25^{♦} | dd^{♦} | 12.5, 4.2^{♦} | H-3′ |
| 3.50 | s | — | H-4′ |
| 3.86 | d | 10.9 | H-14a |
| 3.99 | s | — | OCH₃ |
| 4.18 | d | 10.9 | H-14b |
| 4.33 | q | 6.5 | H-5′ |
| 4.90 | d | 5.3 | H-7 |
| 5.30 | d | 3.4 | H-1′ |
| 6.13 | s | — | OH-9 |
| 7.66 | m | — | H-3 |
| 7.92 | m | — | H-2, H-1 |

*s = singlet, d-doublet, t = triplet, q = quartet, m-multiplet
^{♦}H₂O signal overlaps this resonance; chemical shift was detected in 2D experiments and coupling constants were measured on J-coupled signals.

¹³C NMR chemical shifts*

| δ (ppm) | Carbon | δ (ppm) | Carbon |
|---|---|---|---|
| 17.1 | C-6′ | 99.5 | C-1′ |
| 29.3 | C-2′ | — | C-5a, C-11a^{▾} |
| 33.2 | C-10 | 119.5 | C-1 |
| 36.7 | C-8 | 120.2 | C-3, C-4a |
| 47.2 | C-3′ | 135.0, 136.0 | C-10a, C-6a |
| 57.0 | OCH₃ | 135.1 | C-12a |
| 61.5 | C-14 | 136.7 | C-2 |
| 66.6 | C-5′ | — | C-11, C-6^{▾} |
| 67.2 | C-4′ | 161.1 | C-4 |
| 69.5 | C-7 | — | C-5, C-12^{▾} |
| — | C-9^{▾} | 208.6 | C-13 |

*Carbon chemical shift were detected in the HSQC and GHMBC experiments
^{▾}no correlations were observed in the GHMBC experiment for these carbons

1. A compound which is an anthracycline glycoside of the formula I: 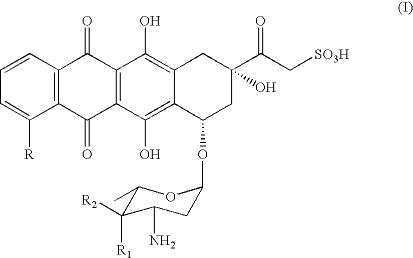
wherein R is hydrogen, hydroxy or methoxy, R₁ and R₂, which are the same or different, are independently hydrogen or hydroxy; or a pharmaceutically acceptable salt thereof.

2. A compound according to claim 1 which is daunorubicin 14-sulfonic acid or 4′epi daunorubicin 14-sulfonic acid.

3. A compound according to claim 1 which is daunorubicin 14-sulfonic acid.

4. A process for producing an anthracycline glycoside of formula (I) as defined in claim 1, which process comprises reacting an anthracycline of the formula II 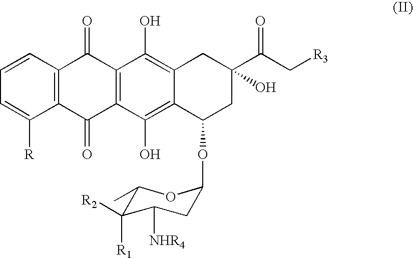
wherein R, R₁ and R₂ are as defined in claim 1, R₃ is hydroxy or a leaving group, and R₄ is hydrogen or a nitrogen protecting group, with a sulfiting agent, in a polar solvent or a mixture of polar solvents and, if desired, removing the protecting groups from the resulting compound.

5. A process according to claim 4 which further comprises converting the resulting compound into a pharmaceutically acceptable salt thereof.

6. A process according to claim 4 or 5 in which the sulfiting agent is sodium sulfite.

7. A process according to any one claims 4 to 6, in which the reaction is carried out at a temperature below 50° C.

8. A process according to claim 7 in which the reaction is carried out at room temperature.

9. A pharmaceutical composition comprising a compound as defined in claim 1, and a pharmaceutically acceptable carrier or diluent.

10. A compound according to any one of claims 1 to 3, for use in a method of treatment of the human or animal body by therapy.

11. A compound as claimed in claim 10 for use as an antitumor agent.

12. Use of a compound as defined in any one of claims 1 to 3 in the manufacture of a medicament for the treatment of tumors.

13. A method of treating a patient in need of an antitumour agent, which method comprises the administration thereto of a compound as defined in any one of claims 1 to 3.
